SEQUENCE LISTING (11 sequences)

SEQ ID NO: 1 - DNA, 11312 bp, Homo sapiens
gtgtctgccaagggtgccagcacagccgccccactccaggggaagaggagtgccagcccttacccacctgagtgggcacagtgtagcatttattcattagcccccacactggcctgaccatctcccctgtgggctgcatgacaaggagagagaacaggctgaggtgagagctactgtcaacacctaaacctaaaaaatctataattgggctgggcagggtggctcacgcctgtaatcccagcactttgggaggccgagatgggtggatcacctgaggtcagatgttcgagaccagcctggccaacatggtgaaaccccgtctctactaaaaatacaaaaaattagctgggcgtggtggtgggtgcctgtaatcccagctactcaggaggctgaggcaggagaattgcttgaacctgggaggcagaggttgcagtgagccgagatcacaccattgcactccagtctgggtgataagtatgaaacgccatctccaaaacaaaagaaaagcctaattccccaagaactgtcagtctttcacctgtctgctagctcccagggagaccccacttgccagggctgtctacatttgtcctgagatctcttctggtgagaacagcactttcctcaggaaagtttgttgaaagtcatcagatccatgattgaaaatcgaagctgcctgtggtgatggataacagctggggttaaaaagcagcagctggggcatgagcggtccacagtgagtttttgttgtttttgtttttttgggtgggggatggggtcttgctaggtctcaaactcctggcctcaagtcatcctcccattacagccttctgagtcactgacactacaggtgtgagccaccatgtccagcttgtagtggttttgaacagctcttgccccttcttgggaatctaggtgccctgcacgtgggtaaggctgtctgcagctgtgcccatattcaggaaggccggcaaggccctgagccctcacacgtgactgacctgaggtgctgtgcagacagcaggtgacggctaagggaaagttgagcactgcctagccgagcactgaagccacgcccggcacacagagagagacccactcggcaaagacttcgcttccaggcacctaaggaactctctgaccagtcattagctgaccactgccgtaactgaagagcggcttcagtggccacagctcgcagggaatggagacattaatgcttagtcagaattagttcagaaaagtcacccagcaaagaaacagctccaacaggcaacaacaacaacacatccttggcagggaagagaatctgacttccggagttgccacattatcgcccgtgaaatgtccaggttttaacaaattatgagacatggaaaggaaaccgaaaggacgacccagacacgggaaaagtcaccaatgggaccagcccgatgctgcaattgctagacaaagacgttcagtcagctcatttaaatatgttcaaagacctaaaacatgctgcatctgaggctgcaccggctggaacctgctgatctcggaagctaagcatggtcaggcctggctagtacttcaaagggagaaaccacgtgtaggcctggtgcagtggctcacacctataatcctagcactctgggaagctgaggcccgtggattgcttgagcccaggagtttgagagcagcttgggaaatgtggtgagacccccatctctacaaaaaaatttaaaaaattagctggctgcctatggtcccagcctctcaggatgctgaggtaggaggatcacttcagcccaggaagttgaggctgcagtgagccatgactgcatcactgcactccagcttgggcgacagagagaccctctcccaagaaaaagaaaagaaccatgtcaaaagaactaacgaaagtgtgggaacaatgtctcaccaattagagaatatcaataatgggatgaaccttataaaaaggggctgggcatggtggctcatgcctataatcccagcactttgggaggctgaggcgggcatatcatgaggtcaagagattgagaccagcctggccaacatggtgaaaccccgtctctacttaaaatacaaaaattagccgggcgtggtggcacgtgcctgtaatcccagctactcgggaggctgaggcaggagaatcgcttgaacccgagaggcagagattgcagtgagccgagattgcaccactgcactacagcctgggtgacagagcgatactccaaaaaacaaaacaaaacaaaaaacaaaaaaaaagtttaaaaaggaaccaaataaaaattctggagttgtagggtaaaataaatgaaaattcatcccaggggcccaagagcagattggaacaattggaagaaagagcctgtgactatggagagaggccacctgaggtagtaccctctgaggaacaggaacaagcatgaagagcaatgcacagagatccagagacctggagacgccgtcaagctttccgacatacacgcaatgggagtcccaggaaagaagacagggagaaaggagtaaaggaatagttgaagaattaatggctgaaaaacctcccaaatctgatgaaaaatattaatctgtacatccaaaaagctcatcaaactccaagtagggtaaactcaaagagatcttcagccatacgcatcatcataatcactgtcaaaagacagatttttctttttttagaattttaaatgtaccttttaatttgctcctggggcaaagagccaggactggtactagagcagtgtctgggatgagaagaatttaataaaatgggattaggtccaatggttgggttaggggaggcaacctgctcgggaggatcagcctcaacctatccatgcagcagggcctccacctgtccctctccgtagtcccacacctggaacccagagccatctgcctcttcccagatcatggccgacagcactccaccagactgcggctggagcaggcacaggattcacttattgagggctgtggcctggcacagatcatagcctatacccagggacagttgtgtcacttctgccaccaccacatccgccttctgcagccacatcaagtaccactcatggatgagcccgtcacccccagcggacttatcaaccccgcgtccagctccacagccgccacgtgctcggtgagcactggctccaagcatggcagctgccatacaatccacctgtagagggcccggtcctcctgtcctcagtggatgatcccgtagaagtccagagctcggcagctgccctcccacaaaagacaggattttgaaagcagcaagagagaagagacgtatcaggtagtcacagtggctcaggcctgtaatcccagcactttgggaggcccaggtgggaggatcgcttcaccccaggaattcaagaccagcctggacaacttggaagaacccggtctctacaaaaaatacaaaattagctgggattgggtgcggtggctcatgcctataatcccagcactttgggagcctgaggtgggtggatcacctgaagtcaggagttcaagactagcctggccaacatggtgaaaccctatctctactgaaaatacaaaaagctagacgtggtggcacacacctgtaatcccagctacttaggaggctgaggcaggagaattgcttgaagcctagaggtgaaggttgtagtgagccgagattgcatcattgcacaatggaggggagccaccagcctgggcaacaagaggaaatctccgtctccaaaaaaaaaaaaaaaaaaaaaagaattaggctgggtggtgcctgtagtcccagctacttgggaggcagggggtccacttgatgtcgagactgcagtgagccatgatcctgccactgcactccggcctgggcaacagagtgagaccctgtctaaagaaaaaaaaaataaagcaacatatcctgaacaaaggatcctccataacgttcccaccagatttctaatcagaaacatggaggccagaaagcagtggaggaggacgaccctcaggcagcccgggaggatgttgtcacaggctggggcaagggccttccggctaccaactgggagctctgggaacagccctgttgcaaacaagaagccatagcccggccagagcccaggaatgtgggctgggctgggagcagcctctggacaggagtggtcccatccaggaaacctccggcatggctgggaagtggggtacttggtgccgggtctgtatgtgtgtgtgactggtgtgtgtgagagagaatgtgtgccctaagtgtcagtgtgagtctgtgtatgtgtgaatattgtctttgtgtgggtgattttctgcgtgtgtaatcgtgtccctgcaagtgtgaacaagtggacaagtgtctgggagtggacaagagatctgtgcaccatcaggtgtgtgcatagcgtctgtgcatgtcaagagtgcaaggtgaagtgaagggaccaggcccatgatgccactcatcatcaggagctctaaggccccaggtaagtgccagtgacagataagggtgctgaaggtcactctggagtgggcaggtgggggtagggaaagggcaaggccatgttctggaggaggggttgtgactacattagggtgtatgagcctagctgggaggtggatggccgggtccactgaaaccctggttatcccagaaggctttgcaggcttcaggagcttggagtggggagagggggtgacttctccgaccaggcccctccaccggcctaccctgggtaagggcctggagcaggaagcaggggcaagaacctctggagcagcccatacccgccctggcctgactctgccactggcagcacagtcaacacagcaggttcactcacagcagagggcaaaggccatcatcagctccctttataagggaagggtcacgcgctcggtgtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcgggccaagaaaccacctgcactagggaggtgtgagcatggggacgagggcggggcttgtgacgagtgggcggggccactgccgagacctggcaggagcccaatgggtgaggctggcgcatttcccagctggaatccggtgtcgaagtggggggcggggaccgcacctgtgctgtaagctcagtgtgggtggcgcggggcccgcggggtcttccctgagtgcaaaggcggtcagggtgggcagagacgaggtggggcaaagccctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcacctggtgatagccccagcatggctactgccaggtgggcccactctaggaaccctggccacctagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaactcgggtcccctgctcacgccctcgttgggatcatcctcctcagggcaaccccacccctgcctcattcctgcttaccccaccgcctggccgcatttgagacaggggtacgttgaggctgagcagatgtcagttacccttgcccataatcccatgtcccccactgacccaactctgactgcccagattggtgacaaggactacattgtcctggcatgtggggaaggggccagaatgggctgactagaggtgtcagtcagccctggatgtggtggagagggcaggactcagcctggaggcccatatttcaggcctaactcagcccaccccacatcagggacagcagtcctgccagcaccatcacaacagtcacctcccttcatatatgacaccccaaaacggaagacaaatcatggcgtcagggagctatatgccagggctacctacctcccagggctcagtcggcaggtgccagaacgttccctgggaaggccccatggaagcccaggactgagccaccaccctcagcctcgtcacctcaccacaggactggctacctctctgggccctcagggatgctgctgtacagacccctgaccagtgacgagttcgcactcagggccaggctggcgctggaggaggacacttgtttggctccaaccctaggtaccatcctcccagtagggatcaggcagggcccacaggcctgccctagggacaggagtcaaccttggacccataaggcactggggcgggcagagaaggaggaggtggcatgggcagctgagagccagagaccctgaccctagtccttgctctgccattaccccgtgtgaccccgggcccacccttccccacccttccccacccttccccaccccgggcttctgtttcccttctgccaacgagaaggctgcttcacctgccccgagtcctgtcttcctgctctgccttctggggctgtggcccttgctggcctggagccccaaccaagggcagggactgctgtcctccacgtctgtcctcaccgacataatgggctgggctgggcacacaggcagtgcccaagagtttctaatgagcatatgattacctgagtcctgggcagaccttcttagggaacagcctgggacagagaaccacagacactctgaggagccacctgaggcctcttttgccagaggaccctacagcctccctggcagcagttccgccagcatttctgtaaatgccctcatgccagggtgcggcccggctgtcagcacgagagggacgttggtctgtcccctggcaccgagtcagtcagaagggtggccagggcccccttgggcccctccagagacaatccactgtggtcacacggctcggtggcaggaagtgctgttcctgcagctgtggggacagggagtgtggatgaagccaggctgggtttgtctgaagacggaggccccgaaaggtggcagcctggcctatagcagcagcaactcttggatttattggaaagattttcttcacggttctgagtcttgggggtgttagaggctcagaaccagtccagccagagctctgtcatgggcacgtagacccggtcccagggcctttgctctttgctgtcctcagaggcctctgcaaagtagaaacaggcagccttgtgagtcccctcctgggagcaaccaaccctccctctgagatgccccggggccaggtcagctgtggtgaaaggtagggatgcagccagctcaggggagtggcccagagttcctgcccacccaaggaggctcccaggaaggtcaaggcacctgactcctgggctgcttccctcccctcccctccccaggtcaggaaggtgggaaagggctggggtgtctgtgaccctggcagtcactgagaagcagggtggaagcagccccctgcagcacgctgggtcagtggtcttaccagatggatacgcagcaacttccttttgaacctttttattttcctggcaggaagaagagggatc

SEQ ID NO: 2 - DNA, 1588 bp, Homo sapiens
atttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaa

SEQ ID NO: 3 - protein, 497 aa, Homo sapiens [sequence omitted]

SEQ ID NO: 4 - DNA, 1587 bp, Homo sapiens
atttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaa

SEQ ID NO: 5 - DNA, 1585 bp, Homo sapiens
atttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaa

SEQ ID NO: 6 - DNA, 1588 bp, Homo sapiens
atttggtagtgaggcaggtatggggctagaagcactggtgcccctggccgtgatagtggccatcttcctgctcctggtggacctgatgcaccggcgccaacgctgggctgcacgctactcaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggttttgggccgcgttcccaaggggtgttcctggcgcgctatgggcccgcgtggcgcgagcagaggcgcttctccgtctccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgaccccatccccctatgagctttgtgctgtgccccgctagaatggggtacctagtccccagcctgctccctagccagaggctctaatgtacaataaagcaatgtggtagttccaa

SEQ ID NO: 7 - DNA, 1588 bp, Homo sapiens
atttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtctccaccttgcgcaacttgggcctgggcaagaagtcgctggagcagtgggtgaccgaggaggccgcctgcctttgtgccgccttcgccaaccactccagacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgtgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgaccccatccccctatgagctttgtgctgtgccccgctagaatggggtacctagtccccagcctgctccctagccagaggctctaatgtacaataaagcaatgtggtagttccaa

SEQ ID NO: 8 - DNA, 1588 bp, Homo sapiens
atttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccatctccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgtgcatagtggtggctgacctgttctctgccgggatggtgaccacctcgaccacgctggcctggggcctcctgctcatgatcctacatccggatgtgcagcgccgtgtccaacaggagatcgacgacatgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgaccccatccccctatgagctttgtgctgtgccccgctagaatggggtacctagtccccagcctgctccctagccagaggctctaatgtacaataaagcaatgtggtagttccaa

SEQ ID NO: 9 - DNA, 1588 bp, Homo sapiens
atttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcgcatgccctgcaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaa

SEQ ID NO: 10 - DNA, 1588 bp, Homo sapiens
atttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcgcatgccctacaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaa

SEQ ID NO: 11 - DNA, 1588 bp, Homo sapiens
atttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctgcaccactgccgtgattcatgaggtgcagcgctttggggacatcgtccccctgggtgtgacccatatgacatcccgtgacatcgaagtacagggcttccgcatccctaagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaa

(19) United States
(12) Patent Application Publication
(10) Pub. No.: US 2023/0167482 A1
(43) Pub. Date: Jun. 1, 2023

(54) FUNCTIONAL CYTOCHROME P450 IN VITRO ASSAY

(71) Applicant: PharmGenetix GmbH, Niederalm, Anif (AT)

(72) Inventors: Charity NOFZIGER, Salzburg (AT); Markus PAULMICHL, Salzburg (AT); Simone VANONI, Salzburg (AT)

(73) Assignee: PharmGenetix GmbH, Niederalm, Anif (AT)

(21) Appl. No.: 17/768,363

(22) PCT Filed: Oct. 14, 2020
(86) PCT No.: PCT/EP2020/078867

§ 371 Date: Apr. 12, 2022

(30) Foreign Application Priority Data
Oct. 14, 2019 (AT) A60228/2019

(51) Int. Cl. C12Q 1/26; C12N 5/10; C12N 5/071; C12N 9/02; C12N 15/52; C12N 15/63

(52) U.S. Cl. CPC C12Q 1/26; C12N 5/10; C12N 5/0686; C12N 9/0079; C12N 15/52; C12N 15/63; C12Y 114/15004

(57) ABSTRACT

An in vitro method for determining the functionality of a cytochrome P450 enzyme variant or an orthologue thereof to metabolize at least one substrate. In particular, the cytochrome P450 enzyme variant is a CYP2D6 variant. Further, a kit including the required reagents and biological material, i.e. cells, to carry out the method.

The present invention provides an in vitro method for determining the functionality of a cytochrome P450 enzyme variant, in particular of a CYP2D6 variant or an orthologue thereof. Further the present invention provides a kit comprising the required reagents (and biological material i.e. cells) to carry out the method disclosed herein.

Based on the principle of pharmacogenomics the method according to the invention uses the information about one individual's genetic information with regard to CYP variants to optimize and personalize medication.

Pharmacogenomics is the study of the role of the genome in drug response. Its name reflects its combining of pharmacology and genomics. Pharmacogenomics analyzes how the genetic makeup of an individual affects his/her response to drugs. It deals with the influence of acquired and inherited genetic variation on drug response in patients by correlating gene expression or single-nucleotide polymorphisms with pharmacokinetics (drug absorption, distribution, metabolism, and elimination) and pharmacodynamics (effects mediated through a drug's biological targets). The term pharmacogenomics is often used interchangeably with pharmacogenetics. Although both terms relate to drug response based on genetic influences, pharmacogenetics focuses on single drug-gene interactions, while pharmacogenomics encompasses a more genome-wide association approach, incorporating genomics and epigenetics while dealing with the effects of multiple genes on drug response.

Pharmacogenomics aims to develop rational means to optimize drug therapy, with respect to the patients' genotype, to ensure maximum efficiency with minimal adverse effects. Through the utilization of pharmacogenomics, it is hoped that pharmaceutical drug treatments can deviate from what is dubbed as the “one-dose-fits-all” approach. Pharmacogenomics also attempts to eliminate the trial-and-error method of prescribing, allowing physicians to take into consideration their patient's genes, the functionality of these genes, and how this may affect the efficacy of the patient's current or future treatments (and where applicable, provide an explanation for the failure of past treatments).

The benefits of pharmacogenetic guided therapy are either improved efficacy, improved safety, reduced hospitalization or reduced cost [30].

Cytochrome P450 (CYP450) is a family of monoxygenases enzymes containing a heme group as cofactor used for oxidation of several molecules, among which steroids, fatty acids and xenobiotics [1]. These enzymes are found in all kingdoms of life, and in mammals they are located mostly in microsomes of hepatocytes or other cell types, like small intestine, lungs, placenta and kidneys [2]. CYP450s are essential for the metabolism of drugs, and although more than 50 isoforms have been identified in this family, only six of them (CYP1A2, CYP2C9, CYP2C19, CYP2D6, CYP3A4 and CYP3A5) metabolize 90% of the existing drugs [2, 3].

Despite accounting for only 1-4% of all hepatic CYP450, CYP2D6 is one of the most important metabolic enzymes, being responsible for the metabolism of around 25% of commonly prescribed drugs, with particular relevance on antidepressants and antipsychotics [4-7]. Although its gene is relatively small (approximately 4.4 kb, containing 9 exons and 8 introns), there are more than 135 unique alleles, defined as star (*) alleles, registered on the human CYP450 allele nomenclature database, Pharmacogene Variation Consortium (PharmVar) [8].

Due to the polymorphic nature of the gene, CYP2D6 enzyme activity ranges widely, resulting in four main different phenotypes: ultrarapid metabolisers (UM), normal metabolisers (NM), intermediate metabolisers (IM) and poor metabolisers (PM) [9]. Among these phenotypes, UMs and PMs have the highest risk for adverse side effects or treatment failure. Considering that in Europe alone, 15-25% of the population are not CYP2D6 NMs [10, 11], the importance of determining the metabolic activity of the enzyme for a targeted treatment is very high. Unpublished data from the inventors' patient cohort (obtained with the inventor's method of a comprehensive CYP2D6 pharmacogenetic assessment) show that the effective NM for CYP2D6 in Europe are <50%. In addition, very few of the known CYP2D6 variants are functionally well-characterized, accordingly to PharmVar [8].

One method to determine CYP2D6 activity is therapeutic drug monitoring (TDM), in which a drug metabolised by CYP2D6 is administered to a patient, and the concentrations of its metabolite in the biological fluid of interest are directly measured [12]. Although patient-specific, this method is only accurate for the drug administered at the very time point in which the bio probe was taken (not allowing for the extrapolation beyond that time point), is laborious, logistically challenging (sample need to be kept on dry ice and analysed within 24 hours), costly and the obtained values differ greatly from laboratory to laboratory and does not take into consideration only CYP2D6 function, but rather the entire metabolic pathway for the administered drug, the contribution of epigenetic factors as well as factors that can not be controlled readily (dietary related inhibition or activation of different CYP systems).

Furthermore, in order to clearly identify the activity of a specific CYP2D6 variant in vivo, the genotype should be informative. This means that the allele of interest either needs to be found in i) homozygosity or ii) in heterozygosity with a complete CYP2D6 deletion (CYP2D6*5). However, most of the CYP2D6 variants are rare; therefore, patients with such informative genotype constellations for TDM studies cannot be easily found. Furthermore, whereas for genetically predicted phenotypes (PM throughout UM) specific dosing advices are available by i.e. CPIC (Clinical Pharmacogenetics Implementation Consortium), no such straight forward dosing advice is available for the Therapeutic drug monitoring (TDM)approach.

Generation of several in silico activity prediction studies was one of the previous strategies used to identify the activity of CYP2D6 alleles [13-15], but problems due to heterogeneous results and lack of knowledge at the level of the CYP2D6 protein structure and conformation led to discussions questioning the reliability of these algorithms.

In the search of a more targeted and reliable system for the analysis of CYP2D6 activity, various in vitro high throughput screening assays have been developed based on the measurement of synthetic or non-synthetic substrate metabolism, either by high-performance liquid chromatography (HPCL) [16, 17], radioactivity [18], fluorescence [19, 20], or a combination thereof [21-23]. Regarding the fluorescence-based assays, one of the major limitations lie in the presence of intrinsic fluorescence or quenching, especially during inhibition studies with compounds derived from natural products, thus leading to over- or underestimation of enzyme activity, respectively [24].

To avoid this problem, second-generation bioluminescence assays were generated [25], among which one involves the use of a methoxy-luciferin-ethylene glycol ester (Luciferin-ME-EGE). Luciferin-ME-EGE is a luciferin derivative that is demethylated by CYP2D6 to obtain luciferin ethylene glycol ester (Luciferin-EGE), a molecule that is then hydrolysed by an esterase to release free luciferin. As a final step, a luciferase enzyme converts the luciferin into a light signal, which is directly proportional to CYP2D6 activity [26]. This system has been used with success for CYP2D6 inhibition studies [27-29], however no extensive study on different CYP2D6 allelic variants has been reported to date. The reason for this is that Luciferin-ME-EGE is not specific for only CYP2D6. Indeed, Luciferin-ME-EGE can also be metabolized by CYP1A1 and CYP1A2, limiting its use for recombinant CYP2D6 coupled with a NADPH regeneration system [26], rather than for in-cell or liver microsomal extract analysis. Given that production of recombinant CYP2D6 for each and every variants would be timely and resource-intensive, it is clear that this system is not ideal for high-throughput analysis.

Furthermore, with the possibility nowadays to easily and quickly carry out sequence analyses, more and more CYP450 variants are identified over time which need to be evaluated with regards to their metabolizing activity. As such, there is clearly a need to quickly obtain functional information about a vast repertoire of CYP450 variants as, for example, CYP2D6 variants.

Thus, the technical problem underlying the present invention is the provision of a method for determining the functionality of CYP450, in particular CYP2D6 variants, in a fast, cost-effective, quantitative, comparable and reliable way.

The solution to the above technical problem is achieved by providing the method characterized in the claims.

### Brief Description of the Sequences of the Invention

It is important to note that the official wild type reference sequences for genes, mRNA transcripts and proteins are given by the National Center for Biotechnology Information (NCBI) database. For the CYP2D6 wild type, these are NG_008376.4 for the gene, NM_000106.6 for the mRNA transcript and NP_000097.3 for the protein. The reference sequence for CYP2D6 has also been assigned an locus reference genome (LRG) number, LRG_303. The Pharmacogene Variation Consortium (PharmVar) is the official pharmacogene nomenclature database, and assigns its own identification numbers, PharmVar IDs (PVIDs) to CYP450 variant alleles that differ from the wild type NCBI reference sequences within a certain set of coordinates that are defined on the PharmVar website (www.pharmvar.org).

SEQ ID NO: 1 corresponds to the nucleic acid sequence of the human wild-type CYP2D6 gene.

SEQ ID NO: 2 corresponds to the nucleic acid sequence of the human wild-type CYP2D6 mRNA transcript.

SEQ ID NO: 3 corresponds to the polypeptide sequence of the human wild-type CYP2D6 protein.

SEQ ID NO: 4 corresponds to the nucleic acid sequence of the human variant CYP2D6*3 (PV00428) mRNA transcript.

SEQ ID NO: 5 corresponds to the nucleic acid sequence of the human variant CYP2D6*9 (PV00434) mRNA transcript.

SEQ ID NO: 6 corresponds to the nucleic acid sequence of the human variant CYP2D6*10

(PV00435) mRNA transcript.

SEQ ID NO: 7 corresponds to the nucleic acid sequence of the human variant CYP2D6*14 (PV00439) mRNA transcript.

SEQ ID NO: 8 corresponds to the nucleic acid sequence of the human variant CYP2D6*29 (PV00453) mRNA transcript.

SEQ ID NO: 9 corresponds to the nucleic acid sequence of the human variant CYP2D6*108 (PV00525) mRNA transcript.

SEQ ID NO: 10 corresponds to the nucleic acid sequence of the human variant CYP2D6*108 (modified 1) mRNA transcript. (c.1074A>G)

SEQ ID NO: 11 corresponds to the nucleic acid sequence of the human variant CYP2D6*108 (modified 1) mRNA transcript. (c.1083A>G)

### BRIEF DESCRIPTION OF THE FIGURES

FIG. 1: Schematic of the protocol for the functional CYP2D6 in vitro assay according to the invention.

FIG. 2: Functional analysis of the CYP2D6*108 (PV00525) variant and two modifications of CYP2D6*108 according to the method of the present invention

FIG. 3: Functional analysis of the CYP2D6*9 (PV00434), CYP2D6*10 (PV00435), CYP2D6*14 (PV00439) and CYP2D6*29 (PV00453) variants according to the method of the present invention

### DESCRIPTION OF THE INVENTION

In a first embodiment, the present invention relates to an in vitro method for determining the functionality of a CYP450 variant or an orthologue thereof to metabolise at least one substrate comprising the steps of:
i) providing human target cells that do not express an enzyme of the cytochrome P450 family (CYP450) or express a non-functional variant of a CYP450 enzyme or express a CYP450 enzyme that is not involved in the metabolism of the substrate used,
ii) transfecting the target cells with at least one nucleic acid encoding at least one CYP450 variant or an orthologue thereof,
iii) culturing the transfected target cells in the presence of at least one substrate under conditions that allow for expression of the CYP450 variant or orthologue thereof and metabolism of the substrate; and
iv) determining the level of CYP450 induced metabolism of the substrate, wherein detection of CYP450 induced metabolism of the substrate is indicative of CYP450 activity.

The term “CYP450 variant” refers to any human CYP450 variant including the wild-type as well as any allele, haplotype, or mutated version thereof. I.e. the method according to the invention can be used for determining the functionality of any CYP450 protein encoded by either the wild-type sequence or amended variants thereof, like certain alleles, haplotypes, mutants etc.

The provided human “target cells” in step i) are the cells to be transfected in step ii). Before being transfected the cells are referred to as “target cells”, whereas after transfection they are referred to as “transfected cells”. The target cells do not comprise any nucleic acid encoding an enzyme of the cytochrome P450 family (CYP450). If the target cells comprise any nucleic acid encoding an enzyme of the cytochrome P450 family (CYP450) the nucleic acid only encodes a non-functional variant of a CYP450 enzyme, which is not able to metabolise a corresponding substrate. Hence, the target cells are not able to express any functional enzyme of the CYP450 family. In other words, the target cells do not endogenously express an enzyme of the cytochrome P450 family. Further, in case the target cells express a functional CYP enzyme it is a CYP enzyme that is not involved in the metabolism of the substrate used, i.e. which is not able to metabolize the used substrate in any way as the CYP450 variant of interest. By this it is ensured that the result measured, i.e. the CYP450 induced metabolism of the substrate, only derives from the CYP450 variant encoded by the nucleic acid that is transfected into the target cell.

It is well known to a person skilled in the art to evaluate whether a cell does not express an enzyme of the cytochrome P450 family (CYP450) or only express a non-functional variant of a CYP450 enzyme or expresses a CYP450 that is not involved in the metabolism of the substrate. An example of how cells can be identified to fulfil these requirements is with sequencing, real-time PCR and/or western blotting, to name a few.

In fact, the usefulness of the target cells needs to be constantly confirmed since the target cells are constantly propagated (undergo division cycles and normal mutation rate). The culture conditions in step ii) are such that they allow for expression of a CYP450 polypeptide. Optimising culture conditions to allow for expression of the polypeptide is well within the routine capabilities of a person of skill in the art. In addition, identifying whether or not the chosen culture conditions allow for polypeptide expression is also routine, and the amount of polypeptide expression may be detected using standard techniques such as ELISA or western blotting.

The culture conditions in step ii) are in the presence of the substrate and are such that they allow for CYP450 induced metabolism of the substrate. In other words, the culture conditions are such that, if a functional CYP450 enzyme were present in the test sample, at least some CYP450 induced metabolism would occur. Optimising culture conditions to allow for CYP450 induced metabolism of the substrate is well within the routine capabilities of a person of skill in the art.

The term “substrate” refers to a molecule upon which an enzyme acts, i.e. for the present invention upon which an enzyme of the CYP450 family acts. In the case of a single substrate, the substrate bonds with the enzyme active site, and an enzyme-substrate complex is formed. The substrate is transformed into one or more products, which are then released from the active site. The active site is then free to accept another substrate molecule.

The substrate may be added to the transfected target cell at the start of cell culture, or may be added later, once polypeptide expression has already commenced. Optimising the time point at which the transfected cell is cultured in the presence of the substrate is well within the routine capabilities of a person of ordinary skill in the art.

Furthermore, it is possible to incubate the cells with more than one substrate wherein the substrates are either all synthetic substrates or all non-synthetic substrates or a mixture of both. By this the functionality of a CYP2D6 variant to metabolize various substrates at the same time or one after the other can be determined.

The term “orthologue” refers to any homologous gene sequence for CYP2D6 found in different species, like for example in horse, cat, dog, etc.

The term “transfection” refers to any method allowing the transfer of nucleic acids into a mammalian cell without any limitation to the actual way of transferring the nucleic acids or the target cell/organism. I.e. the terms “transfection”, “transduction” and “transformation” are used interchangeable herein. Generally, a transfection can be stable or transient. In stable transfection, the plasmid DNA successfully integrates into the cellular genome and will be passed on to future generations of the cell. However, in transient transfection, the transfected material enters the cell but is not integrated into the cellular genome and is therefore not passed onto daughter cells. As stable transfections require a lot of time (several weeks to months) to select and identify the cells in which foreign DNA was successfully integrated into the host genome, and transient transfection can be easily achieved within hours (4-48), the form of transfection for the methods according to the present invention is transient transfection.

This also suits the clinical utility of the described method by allowing to refer the clinician within the time frame necessary for clinical interventions (which would be impossible with the stable transfection approach).

The term “nucleic acid” refers to any kind of nucleic acid sequence encoding for a CYP450 variant. This can, for example, be a single or double stranded nucleic acid sequence or a vector. Preferably the nucleic acid is part of an expression vector (herein referred to as “vector”). Providing a suitable vector for the purposes of the present invention is well within the routine capabilities of a person of ordinary skill in the art.

The term “determining the level of a CYP450 induced metabolism of the substrate” refers to any appropriate method which allows to determine, evaluate or measure the functionality or activity of a CYP450 variant enzyme encoded by the nucleic acid based on the level of the metabolism of the substrate. This is well within the routine capabilities of a person of skill in the art. Thereby the terms “functionality” and “activity” are used interchangeably. By determining the level of the extent of the metabolism of the substrate it can be correlated how functional/active the CYP450 variant is.

In the method according to the invention, the step of determining the functionality of the CYP450 variant comprises the treatment of the transfected cells with at least one CYP450 substrate whereby the degree of metabolism of the substrate, in other words, the level of CYP450 induced metabolism of the substrate, corresponds to the activity of the CYP450 expressed by the nucleic acid or vector with a high degree of metabolism corresponding to a high activity of the CYP450 variant. To test the functionality of the CYP450 variant the cells transfected with the vector encoding the CYP450 variant can be treated with any kind of substrate of interest. Each variant is then binned into one of four categories i) loss of function, ii) decrease of function, iii) normal function and iv) increase of function

The term “non-functional variant of a CYP450 enzyme” refers to any enzyme of the CYP450 family which is not able to metabolize any substrate due to whatever reason. Possible nonfunctional variants are CYP2D6*4 (PV00429), CYP2C9*6 (PV00542), CYP2C19*2 (PV00599), CYP2A13*7(PV00275), CYP2B6*8(PV00746), among others.

The method according to the present invention allows to easily, quickly and reliably test CYP450 variants in a mammalian cell system and obtain information about their functionality with regard to various substrates.

Compared to other eukaryotic cell systems (insect-cells or yeast) used to interrogate the function of CYP enzymes, the present invention has the advantage to determine the CYP-enzyme function in a mammalian-cell system, specifically a human cell system, therefore providing the normal environment in which human enzymes work.

Compared to in vivo methods on humans, the present invention has the advantage of not being invasive and it further allows testing of various substrates without the need of risking a patient's health.

Furthermore, the method according to the invention can be carried out quickly meaning that the required information about the functionality of a CYP450 variant can be obtained within a few days. By this it is possible to decide within a few days which kind of medication can be provided to the patient without risking any side effects or the possibility that the patient will not respond to the provided medication.

A current system for stratifying patients into the four metabolizing phenotype groups (PM throughout UM) is qualitative, rather than quantitative. Currently, CPIC assigns an activity score (AS) to each allele based on its function:

1=fully functional (ie. *1 alleles)
0.5=decreased function (ie. *41 alleles)
0.25=heavily decreased function (*10 alleles)
0=no function (*4 alleles)

The total activity score (TAS) is calculated by summating the AS of each allele a patient has. For example, the TAS of a *1/*1 genotype would be 2 (1 plus 1); the TAS of a *1/*4 genotype would be 1 (1 plus 0); the TAS of a *1/*10 genotype would be 1.25 (1 plus 0.25); and the TAS of a *1/*41 genotype would be 1.5 (1 plus 0.5). TASs of >2.25 are UMs, those between 1.25 and 2.25 are NMs, those between 0 and 1.25 are IMs, and those equal to zero are PMs. In the example from above, the metabolizing phenotypes would be NM for *1/*1, *1/*10 and *1/*41, and IM for *1/*4.

It is obvious that alleles of which the function is unknown cannot be applied to this system. In addition, the more quantitative the functional assessment of a particular allele is, the more precise stratification can be made. Instead of 4 metabolizing groups, 8 (or even more) were able to be distinguished between, therefore augmenting the clinical utility of the method according to the invention. An in vitro system such as the one disclosed here provides such a possibility.

Moreover, since the transfected target cells are human cells, the method according to the present invention provides a system resembling the natural environment of the human CYP450 variant encoded by the nucleic acid. Therefore, there is no need of creating any artificial environment which comes close to the one of the human cell by providing a corresponding NADPH regeneration system, for example. The environment of the human cell allows the encoded CYP450 variant to act as under conditions provided in a human body. Therefore, it is ensured that the functionality/activity of the CYP450 variant is not impaired or changed compared to its functionality/activity in a human body. Hence, the method according to the invention allows to determine the functionality of the encoded CYP450 variant under conditions which are almost identical to those in a human body.

Due to the usage of a target cell line which does not endogenously express any (functional) enzyme of the cytochrome P450 (CYP450) family or if, only a CYP450 enzyme which is not involved in the metabolism of the substrate used, the result of the method will only reflect the functionality of the CYP450 variant transfected into the cell line and will not be influenced by any CYP enzyme activity encoded by the target cell genome.

With the method according to the present invention information about the functionality of a single or several CYP450 variant(s) can be obtained quickly, easily and in a reliable manner allowing to interrogate (i) the specific function of single or multiple genetic variants and/or (ii) substrate specificities thereof, therefore enabling a quick and precise decision about medication possibilities for a patient in need thereof.

Decisions about a patient's medication have to be made in a timely (fast) manner—in a matter a days as opposed to weeks or months, which the current invention would allow for.

The method according to the present invention allows to draw precise conclusion about possible ways of medication for the patent whereas other approaches can bear the risk of choosing the wrong medication due to their experimental set up, like toxicity screenings with cells expressing various CYP enzymes or scoring systems not covering all gene variants or combinations thereof or relying on data based on known enzyme variants although the patient to be treated has a variant that has not been described yet.

Furthermore, with the method according to the present invention databases can be generated easily containing the information about the functionality of the various CYP450 variants which may help to further predict any functionalities of CYP450 variants.

The method according to the invention can help to identify certain subgroups of people based on mutation schemes, or could allow to identify certain parts of the corresponding CYP genes that seem to be responsible for certain levels of enzyme activity. In general, with the method according to the invention it is possible to collect data about certain CYP variants and combinations thereof that can be used of functionality predictions.

The present invention is applicable to any enzyme variant of the CYP450 family.

According to a preferred embodiment of the present invention the CYP450 variant is a CYP2D6 variant.

For example, variant CYP2D6*29 (PV00453) is based on the wild type CYP2D6*1 (PV00126) with the following differences: c.425G>A, c.427G>C, c.905C>T, c.1031G>A, c.1476G>C.

All CYP2D6 variant alleles are officially accepted and catalogued by PharmVar, which includes at this time point the following CYP2D6 variants: CYP2D6*1 (PV00126), CYP2D6*2 (PV00427), CYP2D6*3 (PV00428), CYP2D6*4 (PV00429), CYP2D6*5 (PV00430), CYP2D6*6 (PV00431), CYP2D6*7 (PV00432), CYP2D6*8 (PV00433), CYP2D6*9 (PV00434), CYP2D6*10 (PV00435), CYP2D6*11 (PV00436), CYP2D6*12 (PV00437), CYP2D6*13 (PV00438), CYP2D6*14 (PV00439), CYP2D6*15 (PV00440), CYP2D6*17 (PV00441), CYP2D6*18 (PV00442), CYP2D6*19 (PV00443), CYP2D6*20 (PV00444), CYP2D6*21 (PV00445), CYP2D6*22 (PV00446), CYP2D6*23 (PV00447), CYP2D6*24 (PV00448), CYP2D6*25 (PV00449), CYP2D6*26 (PV00450), CYP2D6*27 (PV00451), CYP2D6*28 (PV00452), CYP2D6*29 (PV00453), CYP2D6*30 (PV00454), CYP2D6*31 (PV00455), CYP2D6*32 (PV00456), CYP2D6*33 (PV00457), CYP2D6*34 (PV00458), CYP2D6*35 (PV00459), CYP2D6*36 (PV00460), CYP2D6*37 (PV00461), CYP2D6*38 (PV00462), CYP2D6*39 (PV00463), CYP2D6*40 (PV00464), CYP2D6*41 (PV00465), CYP2D6*42 (PV00466), CYP2D6*43 (PV00467), CYP2D6*44 (PV00468), CYP2D6*45 (PV00469), CYP2D6*46 (PV00470), CYP2D6*47 (PV00471), CYP2D6*48 (PV00472), CYP2D6*49 (PV00473), CYP2D6*50 (PV00474), CYP2D6*51 (PV00475), CYP2D6*52 (PV00476), CYP2D6*53 (PV00477), CYP2D6*54 (PV00478), CYP2D6*55 (PV00479), CYP2D6*56 (PV00480), CYP2D6*57 (PV00481), CYP2D6*58 (PV00482), CYP2D6*59 (PV00483), CYP2D6*60 (PV00484), CYP2D6*61 (PV00485), CYP2D6*62 (PV00486), CYP2D6*63 (PV00487), CYP2D6*64 (PV00488), CYP2D6*65 (PV00489), CYP2D6*68 (PV00490), CYP2D6*69 (PV00491), CYP2D6*70 (PV00492), CYP2D6*71 (PV00493), CYP2D6*72 (PV00494), CYP2D6*73 (PV00495), CYP2D6*74 (PV00496), CYP2D6*75 (PV00497), CYP2D6*81 (PV00498), CYP2D6*82 (PV00499), CYP2D6*83 (PV00500), CYP2D6*84 (PV00501), CYP2D6*85 (PV00502), CYP2D6*86 (PV00503), CYP2D6*87 (PV00504), CYP2D6*88 (PV00505), CYP2D6*89 (PV00506), CYP2D6*90 (PV00507), CYP2D6*91 (PV00508), CYP2D6*92 (PV00509), CYP2D6*93 (PV00510), CYP2D6*9 (PV00511)4, CYP2D6*95 (PV00512), CYP2D6*96 (PV00513), CYP2D6*97 (PV00514), CYP2D6*98 (PV00515), CYP2D6*99 (PV00516), CYP2D6*100 (PV00517), CYP2D6*101 (PV00518), CYP2D6*102 (PV00519), CYP2D6*103 (PV00520), CYP2D6*104 (PV00521), CYP2D6*105 (PV00522), CYP2D6*106 (PV00523), CYP2D6*107 (PV00524), CYP2D6*108 (PV00525), CYP2D6*109 (PV00526), CYP2D6*110 (PV00527), CYP2D6*111 (PV00528), CYP2D6*112 (PV00529), CYP2D6*113 (PV00530), CYP2D6*114 (PV00531), CYP2D6*115 (PV00532), CYP2D6*116 (PV00533), CYP2D6*117 (PV00534), CYP2D6*118 (PV00535), CYP2D6*119 (PV00536), CYP2D6*120 (PV00630), CYP2D6*121 (PV00631), CYP2D6*122 (PV00632), CYP2D6*123 (PV00633), CYP2D6*124 (PV00634), CYP2D6*125 (PV00681), CYP2D6*126 (PV00682), CYP2D6*127 (PV00635), CYP2D6*128 (PV00683), CYP2D6*129 (PV00684), CYP2D6*130 (PV00685), CYP2D6*131 (PV00686), CYP2D6*132 (PV00687), CYP2D6*133 (PV00688), CYP2D6*134 (PV00689), CYP2D6*135 (PV00690), CYP2D6*136 (PV00691), CYP2D6*137 (PV00692), CYP2D6*138 (PV00722), CYP2D6*139 (PV00728), CYP2D6*140 (PV01361), CYP2D6*141 (PV01362).

This list is not exhaustive and a person skilled in the art is well aware that changes (additions, deletions or other amendments) can occur to these listings but the skilled person will always be able to evaluate whether a CYP450 variant can be considered as being a CYP2D6 variant or not.

For categorizing the functionality of the CYP2D6 variants, i.e. the CYP2D6 induced metabolism of the substrate the following is applied: loss of function CYP2D6 variants have an activity that is not significantly different from CYP2D6*3 (the positive control for a loss of function variant). Normal function variants have an activity that is not significantly different from CYP2D6*1 (positive control for normal function). Increase of function variants have an activity significantly greater than CYP2D6*1. Decrease of function variants have an activity significantly higher than CYP2D6*3 and significantly lower than that of CYP2D6*1.

The method according to the invention can further comprise the step that the target cells are transfected with a vector encoding an excreted luminescence-producing enzyme.

By transfecting the target cells with the vector encoding an excreted luminescence-producing enzyme, the luminescence produced by the excreted enzyme, i.e. of the protein outside of the cell, can be correlated with the success rate of the transfection. If a high luminescence can be measured derived from the protein outside of the cell, it can be assumed that also a high vector load encoding the CYP2D6 variant entered the cell during transfection since both the nucleic acid, preferably a vector, i.e. the one encoding the CYP2D6 variant and the one encoding the excreted luminescence-producing enzyme, are co-transfected, i.e. are transfected in parallel. This allows to make normalization calculations between single cells or cell cultures.

Each variant is then binned into one of four categories i) loss of function, ii) decrease of function, iii) normal function and iv) increase of function. For details please refer to the “Material and Methods” part. Hence, with the information obtained by the method according to the invention it is possible to categorize the functionality of a patient's CYP2D6 variant which can be helpful for making a decision about a patient's medication.

In the method according to the invention the target cells can be transfected with two or more nucleic acids or vectors each encoding at least one CYP2D6 variant or an orthologue thereof.

By transfecting the cells with two or more nucleic acids or vectors each encoding at least one CYP2D6 variant or an orthologue thereof it is possible to better mimic the situation in a human or animal body always encoding two alleles containing at least one CYP2D6 variant, one allele from the mother and one from the father.

Furthermore, it is possible to investigate combinations of CYP2D6 variants in one cell which might reveal any unknown synergistic or antagonistic effects.

It is also possible that the two or more nucleic acids each encode the same CYP2D6 variant or the orthologue thereof to obtain a higher than normal expression rate of the desired variant.

According to the present invention the cells can also be transfected with one nucleic acid or vector which encodes at least two CYP2D6 variants or an orthologue thereof. By transfecting the target cells with one nucleic acid or vector encoding all CYP2D6 variants of interest it is ensured that the target cells have been transfected equally successful with all encoded CYP2D6 variants since they are encoded by one nucleic acid or vector. When transfecting cells with several vectors encoding different genes it cannot be excluded that the transfection of one vector is more successful than the transfection of the other one resulting in different amounts of vectors in the target cells.

A preferred embodiment of the method according to the invention comprises the transfection of the target cells with two vectors each encoding one CYP2D6 variant.

A more preferable embodiment of the method according to the invention comprises the transfection of the target cells with one vector encoding two CYP2D6 variants. By this the situation as in a human body with two CYP2D6 variants encoded by the genome can be obtained with both variants being transfected in the same amounts. This correspondingly applies to the transfection of the cells with a vector encoding the orthologues of the CYP2D6 variants.

All combinations of the amount of nucleic acids or vectors of CYP2D6 variants or orthologues thereof are possible. For example, the cells can be transfected with one vector encoding one, two, three, four, five or more CYP2D6 variants. Another example would be that the cells are transfected with one, two, three, four, five or more vectors each encoding one, two, three, four, five or more CYP2D6 variants.

Since it is known that people can actually have multiple copies of variants, in particular CYP2D6 variants (up to at least 13 copies), transfections with vectors encoding multiple copies or transfections with several vectors encoding multiple copies these variations are encompassed by the method according to the present invention.

In one embodiment according to the invention the orthologue encoded by the vector is an orthologue from horse, cat or dog, among others.

In a preferred embodiment the orthologue is an equine orthologue like CYP2D50 or CYP2D14-like.

A person skilled in the art is aware about the fact that the identification or sequencing of animal genomes might lead to changes in terms of what can actually be considered as an orthologue of the human CYP2D6. Therefore, it might be that even if not listed here another CYP2D6 orthologue than CYP2D50 or CYP2D14-like orthologues should be considered as the equine CYP2D6 orthologue according to this embodiment.

According to the present invention the CYP2D6 variant can be encoded by a nucleotide sequence at least 75% identical to SEQ ID NO:1 or SEQ ID NO:2. The terms “nucleic acid” and “nucleotide sequence” are used interchangeably herein.

As used herein, “sequence identity”, between two nucleotide sequences, indicates the percentage of nucleic acids that are identical between the sequences, preferably over the entire length of the nucleotide sequences as encoded by SEQ ID NO:1 and/or SEQ ID NO:2.

Preferred nucleotide sequences of the invention have a sequence identity of at least 75%, 80% or 85% to the nucleotide sequence as encoded by SEQ ID NO: 1 or have a sequence identity of at least 75%, 80%, 85%, 90% or 95% to SEQ ID NO:2.

For the method according to the invention the target cells can be CYP2D6 deficient human embryonic kidney (HEK) cells or a modified version thereof and/or the target cells do not express a functional CYP450 enzyme variant involved in the metabolism of the substrate. HEK cells are easy to transfect and easy with regard to their handing for cell culturing.

The term “modified version thereof” refers to HEK cells that derive from HEK cells but have been modified like for example HEK-Blue™ cells, which have been engineered to stably express STAT6. The HEK-Blue™ cells purchased by the inventors have a non-functional CYP2D6 genotype (*4/*4). This provides a virtually perfect, human cellular system to test CYP variants without interference from endogenously expressed CYP enzyme. As previously stated, this cell line stably expresses STAT6, the purpose of which was to allow for detection of bioactive human interleukin 4 and human/mouse interleukin 13, a completely distinct line/field of study. Since this cell line has already been genetically modified, and has underwent multiple division cycles in the hands of the inventors, it is not for granted that every commercially available HEK-Blue™ cell line also has this particular CYP2D6 genotype. It is well within the knowledge of a skilled person to evaluate whether a cell can be considered as a modified version of a cell or not.

In a preferred embodiment the target cells can be a modified version of HEK cells that are deficient for functional CYP2D6 variants and optionally which may only express a nonfunctional variant of a CYP2D6 variant.

According to a further embodiment of the present invention the target cells can (endogenously) express CYP2D6*4 or any other complete loss of function CYP2D6 variant allele. The term “complete loss of function” refers to any variant of the CYP2D6 enzyme which is not able to metabolize any substrate. Further examples of complete loss of function CYP2D6 variant enzymes are *3, *5, *7, *15, among others.

An example of cells for the method according to the invention are HEK-Blue™ cells (HEK-Blue™ IL-4/IL-13 cells, Invivogen, Toulouse, France) provided that they fulfil the requirements and do not express an enzyme of the cytochrome P450 family (CYP450) or express a non-functional variant of a CYP450 enzyme or express a CYP450 that is not involved in the metabolism of the substrate. The HEK-Blue™ cells have been tested by the inventors by Sanger sequencing, demonstrating that this cell line only expresses the non-functional CYP2D6 genotype (*4/*4).

By using target cells that are CYP2D6 deficient, and/or only (endogenously) express complete loss of function CYP2D6 variants or if they express a CYP450 enzyme this enzyme is not involved in the metabolism of the substrate used, the method according to the invention allows to determine the functionality of the CYP2D6 variant only that is encoded by the nucleic acid or vector used for the transfection of the target cells. By using these types of target cells, it is avoided that any background of endogenous CYP2D6 impairs the result obtained. Thus, it is ensured that the functionality determined completely derives from the CYP2D6 variant encoded by the vector used for the transfection of the cells. This allows a clear statement about the functionality of one specific CYP2D6 variant to metabolize a specific substrate to be made.

This is in particular important since it cannot be excluded that the expression of more than one CYP2D6 enzyme in a cell will result in a different functionality of the CYP2D6 variant meaning that various CYP2D6 variants might have an impact on each other's functionality. Only when making sure that no other CYP2D6 variant can be responsible for the measured result a precise statement about this one single CYP2D6 variant can be made.

For the method according to the invention the substrate used can be a synthetic or a non-synthetic substrate.

The method according to the invention allows to determine the functionality of the CYP2D6 variant of interest with regard to any CYP2D6 substrate, no matter if it is a synthetic or non-synthetic substrate. Any substrate or potential substrate for CYP2D6 can be used for the method according to the invention and the functionality of the desired CYP2D6 variant can be determined with regard to the chosen substrate.

Furthermore, the different substrates mentioned above can be tested in the presence or absence of drugs metabolized by the respective CYP enzyme, therefore allowing to determine a substrate specificity for the interrogated CYP enzyme and/or in the presence of a mutated variant thereof.

By this a quick screen of a specific CYP2D6 variant can be carried out in order to determine its functionality in view of a whole range of substrates that might be of interest. Hence, within a few days the functionality of a CYP2D6 variant of a patient can be tested for its functionality to metabolize possible substrates that could be of interest for a treatment of the patient. Based on the result a decision can be made which kind of medication can be applied or not.

Examples for synthetic substrates are: 3-[2-(N,N-diethyl-N-methyl-ammonium)ethyl]-7-methoxy-4-methylcoumarin (AMMC), 7-ethoxy-methyloxy-3-cyanocoumarin (EOMCC), methoxy-luciferin-ethylene glycol ester (Luciferin-ME-EGE), [O-methyl-¹⁴C]-dextromethorphan, and a 5′-carboxypropargyl amide probe, among others.

Examples for non-synthetic substrates are: mipramine, amitriptyline, fluoxetine, paroxetine, fluvoxamine, venlafaxine, duloxetine, mianserin, mirtazapine, codeine, tramadol, O-desmethyltramadol, N-desmethyltramadol, oxycodone, hydrocodone, tapentadol, haloperidol, risperidone, perphenazine, thioridazine, zuclopenthixol, iloperidone, aripiprazole, chlorpromazine, levomepromazine, remoxipride, minaprine, tamoxifen, metoprolol, timolol, alpresnolol, carvedilo, bufuralol, nebivolol, propranolol, debrisoquine, flecainide, propafenone, encainide, mexiletine, lidocaine, sparteine, ondansetron, donepezil, pehnformin, tropisetron, amphetamine, methoxyamphetamine, dextromethampthetamine, atomoxetine, chlorphenamine, dexfenfluramine, destromethorphan, metoclopramide, perhexiline, phenacetin, promethazine, m-tyramine, p-tyramine, diltiazem, nifedipine, citalopram, among others.

These listings for synthetic and non-synthetic substrates are non-exhaustive. A person skilled in the art is aware what can be considered as a CYP2D6 substrate. Further, also the use of potential substrates or substrates that have not been identified as such is encompassed by the present invention. This also encompasses substances in foodstuff that are able to interfere with the metabolisms of drugs.

For determining the functionality of a CYP2D6 variant enzyme to metabolize a substrate by the method according to the present invention various possibilities exist to evaluate the functionality which are well known to a person skilled in the art. The CYP2D6-induced metabolism can for example be determined by ELISA, live cell imaging, immunoblot, chromatography, radioactive labelling, fluorescence, luminescence, mass spectrometry, other methods able to identify respective metabolites, etc.

The method according to the present invention can comprise an additional step for determining the functionality of the CYP2D6 variant comprising the contacting of a luminogenic molecule with the encoded CYP2D6 variant or orthologue thereof and with at least one luciferase enzyme to produce a reaction mixture followed by measuring the luminescence of the reaction mixture to determine the activity of the CYP2D6 variant or orthologue thereof.

Therefore, a commercially available second-generation bioluminescence assay utilizing methoxy-luciferin-ethylene glycol ester (Luciferin-ME-EGE) as the CYP2D6 substrate is used. This substrate is metabolized by the CYP2D6 variant resulting in a luciferin product that generates light with a luciferin detection reagent which is added after the enzyme reaction has been completed. Depending on the luminescence measured, the functionality of the CYP2D6 variant can be correlated to a high functionality when a high luminescence is measured, and to a low functionality when a low luminescence is measured.

Another possibility to measure the functionality of the CYP2D6 variant or of the orthologue thereof is by mass spectrometry. Therefore, the transfected cells are treated with a substrate of interest followed by cell lysis. Metabolites are then extracted from the lysates using solid phase extraction columns and separated by High Performance Liquid Chromatography (HPLC). HPLC peaks for each different metabolite are detected using a mass spectrometer and the peak area ratio compared with the peak area ratio of an internal standard of known concentration, to determine metabolite concentration. Depending on the metabolite concentration, the functionality of the CYP2D6 variant can be correlated to a high functionality when high concentrations of metabolites are measured, and to a low functionality when low concentrations are measured. It is important to mention that this combined strategy allows to correlate the measured metabolite concentration (see also TDM) to a defined phenotype (PM to UM) for which dosing advises exists.

Therefore, the present invention allows to quickly and easily test CYP2D6 variants for the functionalities on various substrates. Hence, within a few days, CYP2D6 variants of one or more individuals can be tested easily with regard to their functionality on one or more substrates. This can be of high relevance when deciding about the treatment and, in particular, medication of a patient.

Another embodiment of the present invention is a kit for determining the functionality of a CYP450 variant or an orthologue thereof comprising a) CYP450 deficient cells or a modified version thereof, like HEK-Blue cells, and b) vectors encoding CYP450 control variants.

With such a kit a person skilled in the art is able to carry out a functionality test of a CYP450 variant of interest according to the present invention.

The cells in a) have been described in detail elsewhere herein.

Optionally, the kit further comprises one or several nucleic acid(s) or expression vector(s) encoding the CYP450 variant(s) of interest.

Optionally, the kit further comprises a vector encoding a luminescent excreted protein and reagents for quantification of the luminescent excreted protein.

Optionally, the kit further comprises a (synthetic) substrate for CYP2D6 and reagents for quantification of the metabolized (synthetic) substrate.

In a preferred embodiment of the invention the kit is for determining the functionality of a CYP2D6 variant or an orthologue thereof.

The CYP2D6 control variants can comprise vectors encoding non-functional CYP2D6 variants (e.g. CYP2D6*3 (PV00428), CYP2D6*4 (PV00429), CYP2D6*15 (PV00440), etc.) showing no functionality, the CYP2D6 wild-type (CYP2D6*1 (PV00126)) and/or variants with a decreased functionality (e.g. CYP2D6*9 (PV00434), CYP2D6*10 (PV00435), CYP2D6*17 (PV00441), etc).

Possible CYP2D6 variants of interest that can be part of the kit are CYP2D6*1 (PV00126), CYP2D6*2 (PV00427), CYP2D6*3 (PV00428), CYP2D6*4 (PV00429), CYP2D6*5 (PV00430), CYP2D6*6 (PV00431), CYP2D6*7 (PV00432), CYP2D6*8 (PV00433), CYP2D6*9 (PV00434), CYP2D6*10 (PV00435), CYP2D6*11 (PV00436), CYP2D6*12 (PV00437), CYP2D6*13 (PV00438), CYP2D6*14 (PV00439), CYP2D6*15 (PV00440), CYP2D6*17 (PV00441), CYP2D6*18 (PV00442), CYP2D6*19 (PV00443), CYP2D6*20 (PV00444), CYP2D6*21 (PV00445), CYP2D6*22 (PV00446), CYP2D6*23 (PV00447), CYP2D6*24 (PV00448), CYP2D6*25 (PV00449), CYP2D6*26 (PV00450), CYP2D6*27 (PV00451), CYP2D6*28 (PV00452), CYP2D6*29 (PV00453), CYP2D6*30 (PV00454), CYP2D6*31 (PV00455), CYP2D6*32 (PV00456), CYP2D6*33 (PV00457), CYP2D6*34 (PV00458), CYP2D6*35 (PV00459), CYP2D6*36 (PV00460), CYP2D6*37 (PV00461), CYP2D6*38 (PV00462), CYP2D6*39 (PV00463), CYP2D6*40 (PV00464), CYP2D6*41 (PV00465), CYP2D6*42 (PV00466), CYP2D6*43 (PV00467), CYP2D6*44 (PV00468), CYP2D6*45 (PV00469), CYP2D6*46 (PV00470), CYP2D6*47 (PV00471), CYP2D6*48 (PV00472), CYP2D6*49 (PV00473), CYP2D6*50 (PV00474), CYP2D6*51 (PV00475), CYP2D6*52 (PV00476), CYP2D6*53 (PV00477), CYP2D6*54 (PV00478), CYP2D6*55 (PV00479), CYP2D6*56 (PV00480), CYP2D6*57 (PV00481), CYP2D6*58 (PV00482), CYP2D6*59 (PV00483), CYP2D6*60 (PV00484), CYP2D6*61 (PV00485), CYP2D6*62 (PV00486), CYP2D6*63 (PV00487), CYP2D6*64 (PV00488), CYP2D6*65 (PV00489), CYP2D6*68 (PV00490), CYP2D6*69 (PV00491), CYP2D6*70 (PV00492), CYP2D6*71 (PV00493), CYP2D6*72 (PV00494), CYP2D6*73 (PV00495), CYP2D6*74 (PV00496), CYP2D6*75 (PV00497), CYP2D6*81 (PV00498), CYP2D6*82 (PV00499), CYP2D6*83 (PV00500), CYP2D6*84 (PV00501), CYP2D6*85 (PV00502), CYP2D6*86 (PV00503), CYP2D6*87 (PV00504), CYP2D6*88 (PV00505), CYP2D6*89 (PV00506), CYP2D6*90 (PV00507), CYP2D6*91 (PV00508), CYP2D6*92 (PV00509), CYP2D6*93 (PV00510), CYP2D6*9 (PV00511)4, CYP2D6*95 (PV00512), CYP2D6*96 (PV00513), CYP2D6*97 (PV00514), CYP2D6*98 (PV00515), CYP2D6*99 (PV00516), CYP2D6*100 (PV00517), CYP2D6*101 (PV00518), CYP2D6*102 (PV00519), CYP2D6*103 (PV00520), CYP2D6*104 (PV00521), CYP2D6*105 (PV00522), CYP2D6*106 (PV00523), CYP2D6*107 (PV00524), CYP2D6*108 (PV00525), CYP2D6*109 (PV00526), CYP2D6*110 (PV00527), CYP2D6*111 (PV00528), CYP2D6*112 (PV00529), CYP2D6*113 (PV00530), CYP2D6*114 (PV00531), CYP2D6*115 (PV00532), CYP2D6*116 (PV00533), CYP2D6*117 (PV00534), CYP2D6*118 (PV00535), CYP2D6*119 (PV00536), CYP2D6*120 (PV00630), CYP2D6*121 (PV00631), CYP2D6*122 (PV00632), CYP2D6*123 (PV00633), CYP2D6*124 (PV00634), CYP2D6*125 (PV00681), CYP2D6*126 (PV00682), CYP2D6*127 (PV00635), CYP2D6*128 (PV00683), CYP2D6*129 (PV00684), CYP2D6*130 (PV00685), CYP2D6*131 (PV00686), CYP2D6*132 (PV00687), CYP2D6*133 (PV00688), CYP2D6*134 (PV00689), CYP2D6*135 (PV00690), CYP2D6*136 (PV00691), CYP2D6*137 (PV00692), CYP2D6*138 (PV00722), CYP2D6*139 (PV00728), CYP2D6*140 (PV01361), CYP2D6*141 (PV01362).

Optionally the kit further comprises reagents for the detection of luciferase activity. Suitable reagents are well known in the art and include but are not limited to luciferin, furimazine, coelenterazine, ATP and other known luciferase substrates.

The kits may include any or all of the following: assay reagents, buffers, and selective binding partners for one or more of the metabolized substrate, all of which can be housed in a container suitable for transport.

In addition, the kits may include instructional materials containing directions (i.e., protocols) for the use of the materials provided in the kit. While the instructional materials typically comprise written or printed materials, they may be provided in any medium capable of storing such instructions and communicating them to an end user. Suitable media include, but are not limited to, electronic storage media (e.g., magnetic discs, tapes, cartridges, chips) and optical media (e.g., CD ROM). The media may include addresses to websites that provide the instructional materials. Such instructions may be in accordance with any of the methods or uses detailed herein.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1 94); and Hale and Marham, The Harper Collins Dictionary of Biology, Harper Perennial, N.Y. (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

The inventors have shown that by using the method according to the present invention the functionality of a CYP2D6 variant can be determined in a time-efficient and cost-efficient manner.

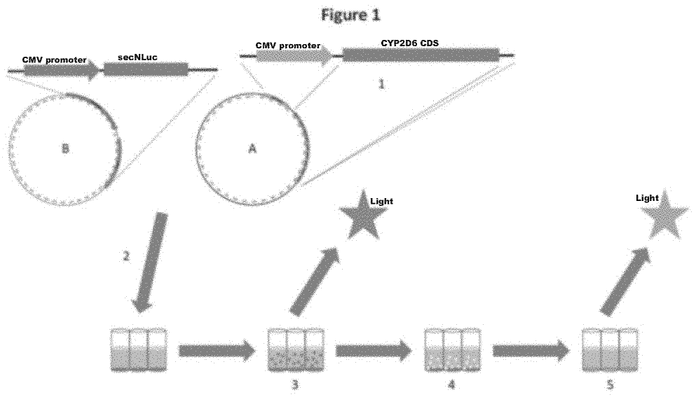

Therefore the CYP2D6 coding sequence (CDS) of interest is cloned into pcDNA3.1-HISC vector, and variants of interest are produced via site-directed mutagenesis (FIG. 1; step 1).

pcDNA3.1-HISC (A) and pNL1.3CMV[secNluc/CMV] (B) vectors are co-transfected in HEK-Blue™ cells (FIG. 1; step 2). 48 hours after transfection, secreted NanoLuc is detected and measured by luminescence using a multi-plate reader (FIG. 1; step 3). Following NanoLuc detection, old media is replaced with Luciferin ME-EGE containing media (FIG. 1; step 4). After 3 hours incubation at 37° C., cells are lysed and luciferin production (proportional to CYP2D6 activity) is measured using a multi-plate reader (FIG. 1; step 5).

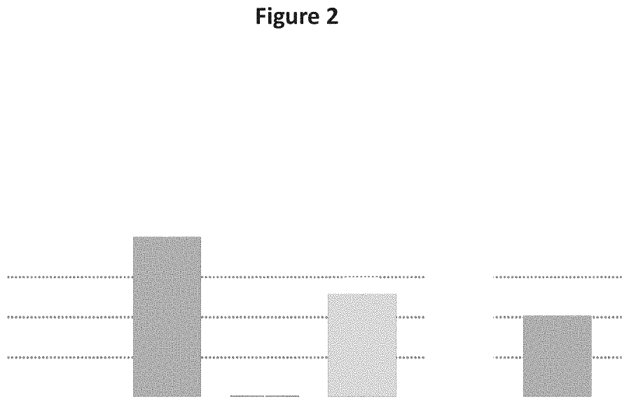

Vectors containing CYP2D6*108 (PV00525) allele, or only one of the two nucleotide variants characterizing it (c.1074A>G and c.1083A>G), were assayed with the method described, along with the no-CYP2D6 (MOCK), positive (*1 (PV00126) -wt; wt=wild type) and negative (*3 (PV00428) -c.794delA; del=deletion) controls (FIG. 2). The *108 allele is shown to have around 65% of the activity with respect of *1, and the nucleotide variation leading to the decreased function is shown to be c.1083A>G (around 50% measured activity), while the c.1074A>G variant is not impacting CYP2D6 activity (98% measured activity). Bars are representing mean values ±SD (Standard deviation) of a total of 24 replicates from 4 different experiments. ****P≤0,0001 as indicated by one-way ANOVA followed by Dunnett post hoctest. ANOVA, analysis of variance; n.s.=not significant.

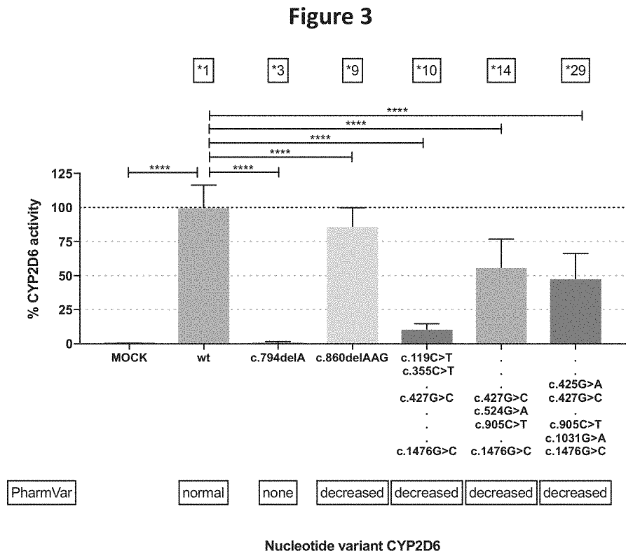

Vectors containing CYP2D6*9 (PV00434), *10 (PV00435), *14 (PV00439) and *29 (PV00453) alleles were assayed with the method described, along with the no-CYP2D6 (MOCK), positive (*1 (PV00126) -wt) and negative (*3 (PV00428) -c.794delA) controls (FIG. 3). These alleles were chosen since their activity was already verified as stated on the Pharmacogene Variation Consortium (PharmVar) website. Being all significantly different than *1 and *3 controls, following categorisation as described in the method section all the four variants matched the known decrease of function activity. Bars are representing mean values ±SD of a total of 24 replicates from 4 different experiments. ****P≤0,0001 as indicated by one-way ANOVA followed by Dunnett post hoc test. ANOVA, analysis of variance.

### Materials and Methods

### Plasmid Preparation and Mutagenesis

CYP2D6 cDNA was obtained from total liver RNA (Ambion®, Life Technology, Wien, Austria), following Reverse-Transcription-PCR (RT-PCR) using the Quantitect® Reverse Transcription kit (Qiagen, Hilden, Germany) following manufacturer instructions. The CYP2D6 coding sequence (CDS) was then amplified using the following oligonucleotide primers: 5′-AATATAAGCTAGCGCCCATTTGGTAGTGAGGCAGGTATGG-3′ (forward primer) and 5′-CTAGGGAGCAGGCTGGGGACTAGG-3′ (reverse primer). The CYP2D6 CDS was then cloned into the pcDNA3.1-HISC eukaryotic protein expression vector under the control of the CMV immediate enhancer and promoter, using the NheI and KpnI restriction digestion enzymes (Thermo Scientific, Waltham, Mass., USA). Site-directed mutageneses of the cloned sequence were performed using the Phusion Site-Directed mutagenesis kit (Thermo Scientific, Waltham, Mass., USA) according to the manufacturer's instructions, in order to obtain fully functional (*1 (PV00126)), complete loss of function (*3 (PV00428)) and decrease of function (*10 (PV00435)) CYP2D6 variants, chosen as controls for the assay. All the other tested CYP2D6 variants were similarly created. After production, vectors and cloned inserts were verified with Sanger sequencing against the wild-type reference sequence (SEQ ID No: 2).

Cell culture and transfection Human Embryonic Kidney (HEK)-Blue (HEK-Blue™ IL-4/IL-13 cells, Invivogen, Toulouse, France) cells, an engineered cell line derived from HEK-293 cells, were cultured in a humidified incubator at 37° C. with 5% CO₂ with Dulbecco's Modified Eagles Medium (DMEM) supplemented with 4.5 g/L glucose, 10% (v/v) Fetal Bovine Serum (FBS), 1.5% NaHCO₃, 50 U/mL penicillin, 50 μg/mL streptomycin, 10 μg/mL Blasticidin and 100 μg/mL Zeocin™ (Invivogen, Toulouse, France). Media was replaced three times weekly.

These cells were chosen since their genotype for CYP2D6 is *4/*4 leading to a fully nonfunctional endogenous CYP2D6. Moreover, further analysis with western blot confirmed that these cells do not express detectable levels of endogenous CYP2D6.

HEK-Blue™ cells were seeded into white 96-well Nunclon™ Δ Surface plates (Thermo Scientific, Waltham, Mass., USA), using a layout of 6 wells for each control or test vector. Twenty-four hours following seeding, cells were transiently co-transfected with the pcDNA3.1-HISC-CYP2D6 constructs and the pNL1.3.CMV [secNluc/CMV] vector (Promega, Mannheim, Germany), by replacing old media with media containing a transfection mix composed of 7.5 mmol/L CaCl₂), 4.2 mmol/L NaCl, 0.045 mmol/L Na₂HPO₄, 1.5 mmol/L HEPES, 0.27 fM pcDNA3.1-HISC vector and 0.038 fM pNL1.3CMV [secNluc/CMV] vector.

The pNL1.3.CMV [secNluc/CMV] plasmid expresses a small luciferase reporter (NanoLuc®) fused to an N-terminal secretion signal under the control of the Human CMV immediate enhancer and promoter. NanoLuc® secretion allows for the normalization of the transfection levels without disturbing the cells.

From this plate, two separate assays were performed: in the first one, aliquots from the supernatant of each well were used for the NanoLuc® detection, while in the second the cells monolayers were used to establish CYP2D6 activity.

### Nano-Glo® Luciferase Assay

Forty-eight hours following transfection, a 20 μL aliquot of the supernatant was transferred from each well into a new white 96-well Nunclon™ A Surface plate for the detection of the secreted NanoLuc® levels using the Nano-Glo® Luciferase assay (Promega, Mannheim, Germany). The supernatant aliquots containing the secreted luciferase were diluted to 1004 for each well using HEK Blue™ cell media and then combined with 100 μL Nano-Glo® Luciferase Assay reagent containing the luminogenic molecule furimazine as NanoLuc® Luciferase substrate. Following three minutes incubation at room temperature (RT), luminescence was measured using the Spark® 10M multimode multiplate reader (Tecan, Grodig, Austria), keeping an optical density filter of 1 (OD1) and 1 second of integration time. Luminescence levels directly correspond to the amount of secreted NanoLuc®, which is used for normalization of transfection efficiency.

### P450-Glo™ Assay

Following secreted NanoLuc® detection, the supernatant was removed and the cells assayed to determine the activity level of the exogenously expressed CYP2D6 variant. Cells were washed once with phosphate buffered saline (PBS). 100 μL of fresh HEK-Blue™ media containing 50 μM Luciferin-ME EGE (Promega, Mannheim, Germany) were then added to each well. Luciferin-ME EGE is a pro-luciferin compound that is converted by CYP2D6 into D-Luciferin. CYP2D6 transfected cells were then incubated for three hours at 37° C. and 5% CO₂ to allow metabolization by the exogenously expressed CYP2D6. At the end of the three hours, 100 μL Luciferin detection reagent (Promega, Mannheim, Germany) were added to each well and the cells incubated for twenty minutes at RT. The light generated is measured using the Spark® 10M multimode multiplate reader (Tecan, Grodig, Austria), with 1 second integration time and no filters. The detected light signal is proportional to D-Luciferin levels and therefore to CYP2D6 activity, and it is normalized over the previously measured secreted NanoLuc® levels accounting for variability in transfection efficiency.

### Categorization of the Evaluated Variants

Activity values of CYP2D6 variants are expressed as percentages relative to that generated by wildtype CYP2D6 (*1). Graphs were made by GraphPad Prism software version 7.0c (GraphPad software, San Diego, Calif.). Each variant is then binned into one of four categories i) loss of function, ii) decrease of function, iii) normal function and iv) increase of function. Loss of function variants have values that are not significantly different from CYP2D6*3 (the positive control for a loss of function variant), Normal function variants have values that are not significantly different from CYP2D6*1 (positive control for normal function). Increase of function variants have values that are significantly greater than CYP2D6*1. Decrease of function variants have values significantly higher than CYP2D6*3 and significantly lower than that of CYP2D6*1.

### REFERENCES

1. Danielson, P. B., The cytochrome P450 superfamily: biochemistry, evolution and drug metabolism in humans. Curr Drug Metab, 2002. 3(6): p. 561-97.
2. Slaughter, R. L. and D. J. Edwards, Recent advances: the cytochrome P450 enzymes. Ann Pharmacother, 1995. 29(6): p. 619-24.
3. Wilkinson, G. R., Drug metabolism and variability among patients in drug response. N Engl J Med, 2005. 352(21): p. 2211-21.
4. Zhou, S. F., Polymorphism of human cytochrome P450 2D6 and its clinical significance: Part I. Clin Pharmacokinet, 2009. 48(11): p. 689-723.
5. Zhou, S. F., Polymorphism of human cytochrome P450 2D6 and its clinical significance: part II. Clin Pharmacokinet, 2009. 48(12): p. 761-804.
6. Ingelman-Sundberg, M., et al., Influence of cytochrome P450 polymorphisms on drug therapies: pharmacogenetic, pharmacoepigenetic and clinical aspects. Pharmacol Ther, 2007. 116(3): p. 496-526.
7. Scordo, M. G. and E. Spina, Cytochrome P450 polymorphisms and response to antipsychotic therapy. Pharmacogenomics, 2002. 3(2): p. 201-18.
8. ; Available from: https://www.pharmvar.org/gene/CYP2D6.
9. Zanger, U. M. and M. Schwab, Cytochrome P450 enzymes in drug metabolism: regulation of gene expression, enzyme activities, and impact of genetic variation. Pharmacol Ther, 2013. 138(1): p. 103-41.
10. Griese, E. U., et al., Assessment of the predictive power of genotypes for the in-vivo catalytic function of CYP2D6 in a German population. Pharmacogenetics, 1998. 8(1): p. 15-26.
11. Gaedigk, A., et al., The CYP2D6 activity score: translating genotype information into a qualitative measure of phenotype. Clin Pharmacol Ther, 2008. 83(2): p. 234-42.
12. Kang, J. S. and M. H. Lee, Overview of therapeutic drug monitoring. Korean J Intern Med, 2009. 24(1): p. 1-10.
13. Martiny, V. Y., et al., Integrated structure- and ligand-based in silico approach to predict inhibition of cytochrome P450 2D6. Bioinformatics, 2015. 31(24): p. 3930-7.
14. Shao, C. Y., et al., CypRules: a rule-based P450 inhibition prediction server. Bioinformatics, 2015. 31(11): p. 1869-71.
15. Jamei, M., et al., The Simcyp population-based ADME simulator. Expert Opin Drug Metab Toxicol, 2009. 5(2): p. 211-23.
16. Yu, A. and R. L. Haining, Comparative contribution to dextromethorphan metabolism by cytochrome P450 isoforms in vitro: can dextromethorphan be used as a dual probe for both CTP2D6 and CYP3A activities? Drug Metab Dispos, 2001. 29(11): p. 1514-20.
17. Paine, M. J., et al., Residues glutamate 216 and aspartate 301 are key determinants of substrate specificity and product regioselectivity in cytochrome P450 2D6. J Biol Chem, 2003. 278(6): p. 4021-7.
18. Delaporte, E., et al., The potential for CYP2D6 inhibition screening using a novel scintillation proximity assay-based approach. J Biomol Screen, 2001. 6(4): p. 225-31.
19. Qu, Q., et al., Inhibitory effects of phytochemicals on metabolic capabilities of CYP2D6(*)1 and CYP2D6(*)10 using cell-based models in vitro. Acta Pharmacol Sin, 2014. 35(5): p. 685-96.
20. Williams, I. S., et al., Biotransformation, Using Recombinant CYP450-Expressing Baker's Yeast Cells, Identifies a Novel CYP2D6.10. ACS Omega, 2018. 3(8): p. 8903-8912.
21. Muroi, Y., et al., Functional characterization of wild-type and 49 CYP2D6 allelic variants for N-desmethyltamoxifen 4-hydroxylation activity. Drug Metab Pharmacokinet, 2014. 29(5): p. 360-6.
22. Sakuyama, K., et al., Functional characterization of 17 CYP2D6 allelic variants (CYP2D6.2, 10, 14A-B, 18, 27, 36, 39, 47-51, 53-55, and 57). Drug Metab Dispos, 2008. 36(12): p. 2460-7.
23. Glass, S. M., et al., CYP2D6 Allelic Variants *34, *17-2, *17-3, and *53 and a Thr309Ala Mutant Display Altered Kinetics and NADPH Coupling in Metabolism of Bufuralol and Dextromethorphan and Altered Susceptibility to Inactivation by SCH 66712. Drug Metab Dispos, 2018. 46(8): p. 1106-1117.
24. Zou, L., M. R. Harkey, and G. L. Henderson, Effects of intrinsic fluorescence and quenching on fluorescence-based screening of natural products. Phytomedicine, 2002. 9(3): p. 263-7.
25. Modarai, M., et al., The interaction potential of herbal medicinal products: a luminescence-based screening platform assessing effects on cytochrome P450 and its use with devil's claw (Harpagophyti radix) preparations. J Pharm Pharmacol, 2011. 63(3): p. 429-38.
26. Cali, J. J., et al., Luminogenic cytochrome P450 assays. Expert Opin Drug Metab Toxicol, 2006. 2(4): p. 629-45.
27. MacArthur, R., et al., Monitoring compound integrity with cytochrome P450 assays and qHTS. J Biomol Screen, 2009. 14(5): p. 538-46.
28. Veith, H., et al., Comprehensive characterization of cytochrome P450 isozyme selectivity across chemical libraries. Nat Biotechnol, 2009. 27(11): p. 1050-5.
29. Hochleitner, J., et al., A combinatorial approach for the discovery of cytochrome P450 2D6 inhibitors from nature. Sci Rep, 2017. 7(1): p. 8071.
30. Krebs and Milani, Translating pharmacogenomics into clinical decisions: do not let the perfect be the enemy of the good. Human Genomics, 2019. 13:39.

1.-19. (canceled)

20. An in vitro method for determining the functionality of a CYP450 variant or an orthologue thereof to metabolize at least one substrate comprising the steps of:
i) providing human target cells that do not express an enzyme of the cytochrome P450 family (CYP450) or express a non-functional variant of a CYP450 enzyme or express a CYP450 that is not involved in the metabolism of the substrate
ii) transiently transfecting the target cells with at least one nucleic acid encoding at least one CYP450 variant or an orthologue thereof,
iii) culturing the transfected target cells in the presence of the at least one substrate under conditions that allow for expression of the CYP450 variant or orthologue thereof and metabolism of the substrate; and
iv) determining the level of CYP450 induced metabolism of the substrate, wherein detection of CYP450 induced metabolism of the substrate is indicative of CYP450 activity.

21. The method according to claim 20, wherein the CYP450 variant is a CYP2D6 variant.

22. The method according to claim 20, wherein the target cells are further transfected with a vector encoding an excreted luminescence producing enzyme.

23. The method according to claim 20, wherein the cells are transfected with two or more nucleic acids each encoding at least one CYP2D6 variant or an orthologue thereof.

24. The method according to claim 20, wherein the cells are transfected with one nucleic acid encoding at least two CYP2D6 variants or an orthologue thereof.

25. The method according to claim 24, wherein the orthologue is an orthologue from the horse, the cat, or the dog, preferably wherein the orthologue is an equine orthologue of CYP2D6.

26. The method according to claim 20, wherein the CYP2D6 variant is encoded by a nucleotide sequence at least 75% identical to SEQ ID No: 1 or SEQ ID NO. 2.

27. The method according to claim 20, wherein the target cells are CYP450 deficient human embryonic kidney (HEK) cells or a modified version thereof and/or wherein the target cells do not express a functional CYP450 enzyme variant involved in the metabolism of the substrate.

28. The method according to claim 20, wherein the target cells are a modified version of HEK cells that are deficient for functional CYP2D6 variants and optionally which express a non-functional variant of a CYP2D6 variant.

29. The method according to claim 20, wherein the target cells express CYP2D6*4 or any other complete loss of function CYP2D6 variant.

30. The method according to claim 20, wherein the cells are incubated with one or more synthetic or non-synthetic substrates or a mixture of synthetic or non-synthetic substrates.

31. The method according to claim 20, further comprising the step of contacting a luminogenic molecule with the encoded CYP2D6 variant or orthologue thereof and with at least one luciferase enzyme to produce a reaction mixture followed by measuring the luminescence of the reaction mixture to determine the functionality of the CYP2D6 variant or orthologue thereof.

32. The method according to claim 20, wherein the functionality of the encoded variant or the orthologue thereof is determined by mass spectrometry.

33. A kit for determining the functionality of at least one CYP450 variant or an orthologue thereof comprising:
a) cells that do not express an enzyme of the cytochrome P450 family (CYP450) or express a non-functional variant of a CYP450 enzyme; and
b) vectors encoding CYP450 control variants.

34. The kit according to claim 33, wherein the kit further comprises c) a vector encoding the CYP450 variant of interest.

35. The kit according to claim 33, wherein the kit further comprises d) a vector encoding a luminescent excreted protein and reagents for quantification of the luminescent excreted protein.

36. The kit according to claim 33, wherein the kit further comprises e) a synthetic or non-synthetic substrate for CYP2D6 and reagents for quantification of the metabolized substrate.

37. The kit according to claim 33, wherein the CYP450 variant to be tested is a CYP2D6 variant and wherein the control variants are CYP2D6 variants.

38. The kit according to claim 37, wherein the CYP2D6 variant is selected from the group consisting of: CYP2D6*1, CYP2D6*2, CYP2D6*3, CYP2D6*4, CYP2D6*5, CYP2D6*6, CYP2D6*7, CYP2D6*8, CYP2D6*9, CYP2D6*10, CYP2D6*11, CYP2D6*12, CYP2D6*13, CYP2D6*14, CYP2D6*15, CYP2D6*17, CYP2D6*18, CYP2D6*19, CYP2D6*20, CYP2D6*21, CYP2D6*22, CYP2D6*23, CYP2D6*24, CYP2D6*25, CYP2D6*26, CYP2D6*27, CYP2D6*28, CYP2D6*29, CYP2D6*30, CYP2D6*31, CYP2D6*32, CYP2D6*33, CYP2D6*34, CYP2D6*35, CYP2D6*36, CYP2D6*37, CYP2D6*38, CYP2D6 *39, CYP2D6*40, CYP2D6 *41, CYP2D6 *42, CYP2D6*43, CYP2D6*44, CYP2D6*45, CYP2D6*46, CYP2D6*47, CYP2D6*48, CYP2D6*49, CYP2D6*50, CYP2D6*51, CYP2D6*52, CYP2D6*53, CYP2D6*54, CYP2D6*55, CYP2D6*56, CYP2D6*57, CYP2D6*58, CYP2D6*59, CYP2D6*60, CYP2D6*61, CYP2D6*62, CYP2D6*63, CYP2D6*64, CYP2D6*65, CYP2D6*68, CYP2D6*69, CYP2D6*70, CYP2D6*71, CYP2D6*72, CYP2D6*73, CYP2D6*74, CYP2D6*75, CYP2D6*81, CYP2D6*82, CYP2D6*83, CYP2D6*84, CYP2D6*85, CYP2D6*86, CYP2D6*87, CYP2D6*88, CYP2D6*89, CYP2D6*90, CYP2D6*91, CYP2D6*92, CYP2D6*93, CYP2D6*94, CYP2D6*95, CYP2D6 *96, CYP2D6 *97, CYP2D6*98, CYP2D6*99, CYP2D6*100, CYP2D6*101, CYP2D6*102, CYP2D6*103, CYP2D6*104, CYP2D6*105, CYP2D6 *106, CYP2D6 *107, CYP2D6 *108, CYP2D6 *109, CYP2D6 *110, CYP2D6*111, CYP2D6 *112, CYP2D6 *113, CYP2D6 *114, CYP2D6 *115, CYP2D6 *116, CYP2D6*117, CYP2D6 *118, CYP2D6 *119, CYP2D6 *120, CYP2D6 *121, CYP2D6 *122, CYP2D6*123, CYP2D6 *124, CYP2D6 *125, CYP2D6 *126, CYP2D6 *127, CYP2D6 *128, CYP2D6*129, CYP2D6 *130, CYP2D6 *131, CYP2D6 *132, CYP2D6 *133, CYP2D6 *134, CYP2D6*135, CYP2D6 *136, CYP2D6 *137, CYP2D6 *138, CYP2D6 *139, CYP2D6 *140, and CYP2D6 *141.
